SEQUENCE LISTING (62 sequences)

SEQ ID NO: 1 - RNA, 23 nt, Homo sapiens
uaaagugcuuauagugcagguag

SEQ ID NO: 2 - RNA, 22 nt, Homo sapiens
uaaggugcaucuagugcagaua

SEQ ID NO: 3 - RNA, 23 nt, Homo sapiens
ugugcaaaucuaugcaaaacuga

SEQ ID NO: 4 - RNA, 24 nt, Homo sapiens
caaagugcuuacagugcagguagu

SEQ ID NO: 5 - RNA, 23 nt, Homo sapiens
ugugcaaauccaugcaaaacuga

SEQ ID NO: 6 - RNA, 22 nt, Homo sapiens
gugaaauguuuaggaccacuag

SEQ ID NO: 7 - RNA, 22 nt, Homo sapiens
uagcuuaucagacugauguuga

SEQ ID NO: 8 - RNA, 23 nt, Homo sapiens
uggcagugucuuagcugguuguu

SEQ ID NO: 9 - RNA, 22 nt, Homo sapiens
aacauucauugcugucgguggg

SEQ ID NO: 10 - RNA, 23 nt, Homo sapiens
uagcaccauuugaaaucaguguu

SEQ ID NO: 11 - RNA, 22 nt, Homo sapiens
cagugcaaugaugaaagggcau

SEQ ID NO: 12 - RNA, 22 nt, Homo sapiens
uucaacggguauuuauugagca

SEQ ID NO: 13 - RNA, 21 nt, Homo sapiens
uaaagugcugacagugcagau

SEQ ID NO: 14 - RNA, 22 nt, Homo sapiens
aaagugcuguucgugcagguag

SEQ ID NO: 15 - RNA, 22 nt, Homo sapiens
cauugcacuugucucggucuga

SEQ ID NO: 16 - RNA, 22 nt, Homo sapiens
uuuggcaaugguagaacucaca

SEQ ID NO: 17 - RNA, 22 nt, Homo sapiens
uuuggcacuagcacauuuuugc

SEQ ID NO: 18 - RNA, 22 nt, Homo sapiens
uauggcacugguagaauucacu

SEQ ID NO: 19 - RNA, 21 nt, Homo sapiens
uagcaccaucugaaaucgguu

SEQ ID NO: 20 - RNA, 20 nt, Homo sapiens
ggcaagaugcuggcauagcu

SEQ ID NO: 21 - RNA, 23 nt, Homo sapiens
aaaagugcuuacagugcagguag

SEQ ID NO: 22 - RNA, 21 nt, Homo sapiens
caagucacuagugguuccguu

SEQ ID NO: 23 - RNA, 22 nt, Homo sapiens
uguaaacauccucgacuggaag

SEQ ID NO: 24 - RNA, 22 nt, Homo sapiens
cuuucagucggauguuugcagc

SEQ ID NO: 25 - RNA, 22 nt, Homo sapiens
cuggacuuggagucagaaggcc

SEQ ID NO: 26 - RNA, 22 nt, Homo sapiens
cuggacuuggagucagaaggcc

SEQ ID NO: 27 - RNA, 21 nt, Homo sapiens
ugagaugaagcacuguagcuc

SEQ ID NO: 28 - RNA, 23 nt, Homo sapiens
guccaguuuucccaggaaucccu

SEQ ID NO: 29 - RNA, 22 nt, Homo sapiens
uacccuguagaaccgaauuugu

SEQ ID NO: 30 - RNA, 23 nt, Homo sapiens
uguaaacauccuacacucucagc

SEQ ID NO: 31 - RNA, 23 nt, Homo sapiens
ucccugagacccuuuaaccugug

SEQ ID NO: 32 - RNA, 21 nt, Homo sapiens
uggaauguaaagaaguaugua

SEQ ID NO: 33 - RNA, 22 nt, Homo sapiens
uugguccccuucaaccagcugu

SEQ ID NO: 34 - RNA, 21 nt, Homo sapiens
cagcagcacacugugguuugu

SEQ ID NO: 35 - RNA, 21 nt, Homo sapiens
uagcagcacagaaauauuggc

SEQ ID NO: 36 - RNA, 21 nt, Homo sapiens
cuggacuuagggucagaaggc

SEQ ID NO: 37 - RNA, 18 nt, Homo sapiens
ucuacagugcacgugucu

SEQ ID NO: 38 - RNA, 22 nt, Homo sapiens
uggaagacuagugauuuuguug

SEQ ID NO: 39 - RNA, 22 nt, Homo sapiens
cacccguagaaccgaccuugcg

SEQ ID NO: 40 - DNA, 21 bp, Artificial Sequence
  Totally synthetic probe designed to complement homo sapien miR-1.
tacatacttctttacattcca

SEQ ID NO: 41 - DNA, 21 bp, Artificial Sequence
  Totally synthetic probe designed to complement homo sapien miR-126
gcattattactcacggtacga

SEQ ID NO: 42 - DNA, 23 bp, Artificial Sequence
  Totally synthetic probe designed to complement homo sapien miR-19a
tcagttttgcatagatttgcaca

SEQ ID NO: 43 - DNA, 23 bp, Artificial Sequence
  Totally synthetic probe designed to complement homo sapien miR-221
gaaacccagcagacaatgtagct

SEQ ID NO: 44 - DNA, 22 bp, Artificial Sequence
  Totally synthetic probe designed to complement homo sapien miR-96
gcaaaaatgtgctagtgccaaa

SEQ ID NO: 45 - DNA, 22 bp, Artificial Sequence
  Totally synthetic probe designed to complement homo sapien miR-182
tgtgagttctaccattgccaaa

SEQ ID NO: 46 - DNA, 24 bp, Artificial Sequence
  Totally synthetic probe designed to complement homo sapien miR-145
aagggattcctgggaaaactggac

SEQ ID NO: 47 - DNA, 21 bp, Artificial Sequence
  Totally synthetic probe designed to complement homo sapien miR-30b
gctgagtgtaggatgtttaca

SEQ ID NO: 48 - DNA, 22 bp, Artificial Sequence
  Totally synthetic probe designed to complement homo sapien miR-194
tccacatggagttgctgttaca

SEQ ID NO: 49 - DNA, 23 bp, Artificial Sequence
  Totally synthetic probe designed to complement homo sapien miR-181a
actcaccgacagcgttgaatgtt

SEQ ID NO: 50 - DNA, 22 bp, Artificial Sequence
  Totally synthetic probe designed to complement homo sapien miR-143
tgagctacagtgcttcatctca

SEQ ID NO: 51 - DNA, 22 bp, Artificial Sequence
  Totally synthetic probe designed to complement homo sapien miR-26a
agcctatcctggattacttgaa

SEQ ID NO: 52 - DNA, 22 bp, Artificial Sequence
  Totally synthetic probe designed to complement homo sapien miR-27a
ggcggaacttagccactgtgaa

SEQ ID NO: 53 - DNA, 23 bp, Artificial Sequence
  Totally synthetic probe designed to complement homo sapien miR-103
tcatagccctgtacaatgctgct

SEQ ID NO: 54 - DNA, 21 bp, Artificial Sequence
  Totally synthetic probe designed to complement homo sapien miR-7
aacaaaatcactagtcttcca

SEQ ID NO: 55 - DNA, 22 bp, Artificial Sequence
  Totally synthetic probe designed to complement homo sapien let-7a
aactatacaacctactacctca

SEQ ID NO: 56 - DNA, 23 bp, Artificial Sequence
  Totally synthetic probe designed to complement homo sapien miR-20a
ctacctgcactataagcacttta

SEQ ID NO: 57 - DNA, 21 bp, Artificial Sequence
  Totally synthetic probe designed to complement homo sapien miR-25
tcagaccgagacaagtgcaat

SEQ ID NO: 58 - DNA, 22 bp, Artificial Sequence
  Totally synthetic probe designed to complement homo sapien miR-125b
tcacaagttagggtctcaggga

SEQ ID NO: 59 - DNA, 22 bp, Artificial Sequence
  Totally synthetic probe designed to complement homo sapien miR-155
cccctatcacgattagcattaa

SEQ ID NO: 60 - DNA, 22 bp, Artificial Sequence
  Totally synthetic probe designed to complement homo sapien miR-100
cacaagttcggatctacgggtt

SEQ ID NO: 61 - DNA, 20 bp, Artificial Sequence
  Totally synthetic U6 snRNA oligonucleotide probe
aacgcttcacgaatttgcgt

SEQ ID NO: 62 - RNA, 18 nt, Homo sapiens
ugcuuccuuucagagggu

(19) United States
(12) Patent Application Publication
(10) Pub. No.: US 2010/0075304 A1
(43) Pub. Date: Mar. 25, 2010

(54) PROCESS FOR MONITORING COLORECTAL CANCER

(76) Inventors: Mitch Raponi, Media, PA (US); Gregory M. Arndt, Sydney (AU)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/261,885

(22) Filed: Oct. 30, 2008

Related U.S. Application Data
(60) Provisional application No. 60/983,771, filed on Oct. 30, 2007.

(51) Int. Cl. C12Q 1/68

(52) U.S. Cl. 435/6

(57) ABSTRACT

Disclosed in this specification is a method for determining the stage of colorectal cancer by observing regulatory changes in select miRNA sequences. These sequences may include hsa-miR-31, hsa-miR-7, hsa-miR-99b, hsa-miR-378*, hsa-miR-133a, hsa-miR-125a and combinations of these sequences.

### CROSS-REFERENCE TO RELATED APPLICATIONS

This application claims priority to and the benefit of co-pending U.S. provisional patent application Ser. No. 60/983,771, filed Oct. 30, 2007, which application is incorporated herein by reference in its entirety.

### REFERENCE TO A SEQUENCE LISTING, A TABLE, OR A PROGRAM LISTING

This application refers to a “Sequence Listing” listed below, which is provided as an electronic document entitled “Sequence3035191.txt” (9 kb, created on Oct. 30, 2008), which is incorporated herein by reference in its entirety.

### FIELD OF THE INVENTION

This invention relates, in one embodiment, to a method for detecting and/or monitoring colorectal cancer (CRC) by observing regulatory changes in the production of select microRNA (miRNA) sequences. By observing up regulation or down regulation changes of specified sequences, both the presence of cancer cells as well as the stage of cancer may be determined.

### BACKGROUND OF THE INVENTION

Colorectal cancer (CRC) is one of the most frequent cancers and a common cause of cancer-related deaths in the developed world. The overall incidence of CRC is 5% in the general population and the 5-year survival rate ranges from 40% to 60%. Prognosis largely relies upon descriptive staging systems using morphology and histopathology of the tumor. However, even morphologically similar tumors can differ in their underlying molecular changes and tumorigenic potential. The development of CRC from normal epithelial cells to malignant carcinomas involves a multi-step process with accumulation of both genetic and epigenetic changes, leading to a temporal activation of oncogenes and inactivation of tumor suppressor genes that confer a selective advantage to cells containing these alterations.

A number of CRC expression profiling studies on protein coding genes have been performed to better resolve the underlying molecular pathways and to further dissect the different stages of CRC. More recently, a newly discovered class of short 22 nucleotide (nt) non-coding RNAs, called microRNAs (miRNAs), have been identified and implicated in cancer initiation and progression. The biogenesis of these small RNAs involves transcription by RNA polymerase II and processing of the primary transcript by the endonuclease Drosha to produce 60-70-nt precursor miRNAs (pre-miRNAs) with imperfect hairpin structures. The pre-miRNA is transported into the cytoplasm through exportin 5 where it undergoes processing by the RNAse III enzyme Dicer to produce mature miRNAs that are then incorporated into a multiprotein complex. These miRNA-containing complexes have been shown to bind to the 3′ untranslated region (UTR) of multiple mRNAs through complementarity between the resident miRNA strand and the target sequence and, based on the degree of homology, direct either translational inhibition or mRNA degradation. To date, there have been 678 human miRNAs identified (miRBase Sequence Database—Release 11) and, through computational models, it has been suggested that there may be greater than 1000 miRNA genes, comprising approximately 3% of the currently known genes in the human genome. Moreover, bioinformatic analyses estimate that miRNAs may regulate as many as 30% of the human protein coding genes, suggesting that these small RNAs may act to coordinate the interplay between complex signal transduction pathways.

Several miRNAs have been identified as differentially expressed between normal and tumor tissues or cancer cell lines. (Calin, G. A. and Croce, C. M. MicroRNA signatures in human cancers. Nat Rev Cancer, 6: 857-866, 2006; Bandres, E., Cubedo, E., Agirre, X., Malumbres, R., Zarate, R., Ramirez, N., Abajo, A., Navarro, A., Moreno, I., Monzo, M., and Garcia-Foncillas, J. Identification by Real-time PCR of 13 mature microRNAs differentially expressed in colorectal cancer and non-tumoral tissues. Mol Cancer, 5: 29, 2006; Cummins, J. M., He, Y., Leary, R. J., Pagliarini, R., Diaz, L. A., Jr., Sjoblom, T., Barad, O, Bentwich, Z., Szafranska, A. E., Labourier, E., Raymond, C. K., Roberts, B. S., Juhl, H., Kinzler, K. W., Vogelstein, B., and Velculescu, V. E. The colorectal microRNAome. Proc Natl Acad Sci USA, 103: 3687-3692, 2006; Michael, M. Z., S M, O. C., van Hoist Pellekaan, N. G., Young, G. P., and James, R. J. Reduced accumulation of specific microRNAs in colorectal neoplasia. Mol Cancer Res, 1: 882-891, 2003; Lanza, G., Ferracin, M., Gafa, R., Veronese, A., Spizzo, R., Pichiorri, F., Liu, C. G., Calin, G. A., Croce, C. M., and Negrini, M. mRNA/microRNA gene expression profile in microsatellite unstable colorectal cancer. Mol Cancer, 6: 54, 2007.)

In CRC, there have been limited studies examining the expression patterns of miRNAs. The first study showing de-regulation of miRNAs reported the down-regulation of miR-143 and miR-145 as early as the pre-adenomatous polyp stage, suggesting a possible role for these miRNAs in early stages of CRC. Subsequently, a group of 13 miRNAs showing differential expression in CRC tumors was identified with the expression level of miR-31 being correlated with CRC tumor stage.

### SUMMARY OF THE INVENTION

The invention comprises, in one form thereof, a method for detecting the presence of colorectal cancer in a cell sample. In another form, the invention is a method for diagnosing the stage of colorectal cancer in a cell sample. Applicants have discovered certain miRNAs that are differentially regulated in colorectal cancers relative to wild type cells. By determining the degree of regulatory changes in such miRNAs, one can determine if a tissue sample includes colorectal cancer cells. Applicants have discovered certain other miRNAs that are differentially regulated in late stage (stage III and IV) colorectal cancers relative to early stage (stage I and II) colorectal cancers. By monitoring these miRNAs, one can differentiate a late stage tumor sample from an early stage tumor sample without needing to rely on less dependable identifiers, such as cell morphology.

### DETAILED DESCRIPTION

### Definitions

The phrase “regulation change” refers to a change in the abundance of a cellular component, such a miRNA, relative to the abundance of the same cellular component in a wild type cell. The phrase “down regulation” refers to a decrease in the abundance of the cellular component in question while the phrase “up regulation” refers to an increase in the abundance of the component.

Identification of Colorectal Cells by Differential miRNA Regulation

Thirty seven differentially expressed miRNAs were identified when colorectal cancer tissue levels were compared to wild type tissue (Table 1). Cell samples including both cell lines and clinical samples, were obtained from commercial sources. Total RNA was extracted from the cell sample in accordance with conventional techniques. For example, mirVana isolation kit (Ambion) for snap-frozen samples and the RecoverAll™ Total Nucleic Acid Isolation Kit for FFPE samples (Ambion) may be used. Other conventional RNA extraction methods may also be used. Once the total RNA is extracted, small (less than forty nucleotides) RNA may be isolated by gel electrophoresis. The samples were analyzed to determine the identity and abundance of specific miRNA sequences. Any suitable technique may be used to determine the identity and abundance such as, but not limited to, Northern blot analysis. Thirty seven differentially expressed miRNAs were identified in colorectal cells which had significantly altered expression relative to a wild type colorectal sample.

TABLE 1
miRNAs differentially expressed between CRC and normal colorectal
tissue.

| | | | | | fold |
|---|---|---|---|---|---|
| SEQ ID. | miRNA | Normal^{a} | CRC^{a} | p-value | change^{b} |
| SEQ ID NO. 1 | hsa-miR-20a | 9.2 | 10.3 | 2.0E−03 | 2.1 |
| SEQ ID NO. 2 | hsa-miR-18a | 7.6 | 8.6 | 2.9E−03 | 2.0 |
| SEQ ID NO. 3 | hsa-miR-19a | 7.7 | 8.7 | 2.3E−03 | 1.9 |
| SEQ ID NO. 4 | hsa-miR-17-5p | 10.5 | 11.4 | 1.5E−03 | 1.9 |
| SEQ ID NO. 5 | hsa-miR-19b | 11.0 | 11.8 | 3.4E−03 | 1.8 |
| SEQ ID NO. 6 | hsa-miR-203 | 8.4 | 9.7 | 2.2E−02 | 2.6 |
| SEQ ID NO. 7 | hsa-miR-21 | 13.0 | 14.5 | 6.0E−06 | 2.9 |
| SEQ ID NO. 8 | hsa-miR-34a | 9.5 | 10.3 | 1.5E−02 | 1.7 |
| SEQ ID NO. 9 | hsa-miR-181b | 8.5 | 9.2 | 2.2E−04 | 1.7 |
| SEQ ID NO. 10 | hsa-miR-29b | 9.7 | 10.5 | 4.9E−03 | 1.8 |
| SEQ ID NO. 11 | hsa-miR-130b | 7.1 | 7.8 | 1.2E−03 | 1.7 |
| SEQ ID NO. 12 | hsa-miR-95 | 6.1 | 6.8 | 1.1E−02 | 1.6 |
| SEQ ID NO. 13 | hsa-miR-106b | 9.5 | 10.3 | 1.0E−04 | 1.7 |
| SEQ ID NO. 14 | hsa-miR-93 | 9.9 | 10.5 | 3.4E−03 | 1.6 |
| SEQ ID NO. 15 | hsa-miR-25 | 9.0 | 9.6 | 1.4E−02 | 1.6 |
| SEQ ID NO. 16 | hsa-miR-182 | 7.2 | 8.7 | 2.8E−03 | 2.8 |
| SEQ ID NO. 17 | hsa-miR-96 | 6.7 | 7.7 | 8.4E−03 | 2.0 |
| SEQ ID NO. 18 | hsa-miR-183 | 5.9 | 6.8 | 2.3E−03 | 1.8 |
| SEQ ID NO. 19 | hsa-miR-29a | 12.1 | 12.9 | 3.6E−04 | 1.7 |
| SEQ ID NO. 20 | hsa-miR-31 | 6.4 | 8.7 | 2.6E−03 | 5.0 |
| SEQ ID NO. 21 | hsa-miR-106a | 10.7 | 11.7 | 5.1E−04 | 2.0 |
| SEQ ID NO. 22 | hsa-miR-224 | 6.9 | 8.4 | 1.1E−03 | 2.8 |
| SEQ ID NO. 23 | hsa-miR-30a- | 11.6 | 11.0 | 1.6E−05 | 0.7 |
| | 5p | | | | |
| SEQ ID NO. 24 | hsa-miR-30a- | 7.1 | 6.3 | 7.3E−04 | 0.5 |
| | 3p | | | | |
| SEQ ID NO. 25 | hsa-miR-378* | 7.6 | 6.5 | 1.3E−05 | 0.4 |
| SEQ ID NO. 26 | hsa-miR-422b | 10.9 | 9.6 | 8.3E−05 | 0.4 |
| SEQ ID NO. 27 | hsa-miR-143 | 14.1 | 12.6 | 1.0E−02 | 0.4 |
| SEQ ID NO. 28 | hsa-miR-145 | 15.0 | 13.2 | 1.2E−03 | 0.3 |
| SEQ ID NO. 29 | hsa-miR-10b | 11.0 | 10.1 | 2.1E−02 | 0.5 |
| SEQ ID NO. 30 | hsa-miR-30c | 10.9 | 10.3 | 4.0E−04 | 0.7 |
| SEQ ID NO. 31 | hsa-miR-125a | 11.2 | 10.2 | 6.4E−03 | 0.5 |
| SEQ ID NO. 32 | hsa-miR-1 | 9.0 | 7.0 | 1.6E−03 | 0.2 |
| SEQ ID NO. 33 | hsa-miR-133a | 10.0 | 7.6 | 6.5E−04 | 0.2 |
| SEQ ID NO. 34 | hsa-miR-497 | 9.3 | 8.1 | 9.8E−04 | 0.4 |
| SEQ ID NO. 35 | hsa-miR-195 | 11.1 | 9.5 | 4.2E−05 | 0.3 |
| SEQ ID NO. 36 | hsa-miR-422a | 10.0 | 8.8 | 7.3E−05 | 0.4 |
| SEQ ID NO. 37 | hsa-miR-139 | 7.6 | 6.2 | 1.1E−05 | 0.4 |

^{a}normalized median signal intensity (log2)
^{b}cancer:normal
Fold Change = 2^{(CRC-Normal)}

Hierarchical clustering showed that many of the above identified miRNA sequences were coordinately expressed, including the miR-143 to −145 and miR17-92 clusters, which were consistently down regulated in CRC samples. It has been observed that the down regulation in both miR-143 and miR-145 is pronounced and their down regulation is consistent over a wide range of cell lines and clinical samples. Accordingly, observing regulatory changes in these two miRNA sequences serves as an indicator to detect the presence of colorectal cancer. The prior art indicates that miR-145 has tumor suppressor effects (Akao et al. Oncology Reports 16: 845-850, 2006; Schepeler et al. Cancer Res. 68 (15), 2008) and are down regulated in CRC cells. However, Applicant has discovered that miR-145 only has tumor suppressor effects in non-metastatic cells and it is actually oncogenic in the metastatic environment. Therefore, the delivery of tumor suppressing hsa-miR-143, without the addition of hsa-miR-145, is a therapeutic strategy for CRC.

In one embodiment of the invention, miRNAs from Table 1 are observed in a biological sample and compared to a wild type (non-cancerous) sample. An un-expected change in abundance (up regulation or down regulation) may be indicative of cancer. The sample may be obtained from a tissue sample or, alternatively, may be obtained non-invasively from a non-tissue sample. For example, a blood, stool, or urine sample may be tested for free miRNAs. In this fashion, screening for CRC is made more convenient. Table 2 shows those miRNA sequences that have been found to be differentially regulated in early stage CRC samples as compared to wild type tissue. Such miRNAs pertain screening to be performed to detect early onset of CRC.

TABLE 2
miRNAs differentially expressed between normal colon and early stage
colorectal cancer (Stages I and II).

| SEQ ID. | Sample ID | Normal | Stage I/II | Fold change |
|---|---|---|---|---|
| SEQ ID NO. 22 | hsa-miR-224 | 6.89 | 8.67 | 3.45 |
| SEQ ID NO. 7 | hsa-miR-21 | 13.02 | 14.49 | 2.78 |
| SEQ ID NO. 8 | hsa-miR-34a | 9.48 | 10.43 | 1.92 |
| SEQ ID NO. 21 | hsa-miR-106a | 10.68 | 11.57 | 1.85 |
| SEQ ID NO. 2 | hsa-miR-18a | 7.51 | 8.38 | 1.83 |
| SEQ ID NO. 10 | hsa-miR-29b | 9.70 | 10.56 | 1.82 |
| SEQ ID NO. 5 | hsa-miR-19b | 10.84 | 11.69 | 1.80 |
| SEQ ID NO. 13 | hsa-miR-106b | 9.45 | 10.26 | 1.76 |
| SEQ ID NO. 1 | hsa-miR-20a | 9.28 | 10.06 | 1.72 |
| SEQ ID NO. 19 | hsa-miR-29a | 12.14 | 12.89 | 1.68 |
| SEQ ID NO. 9 | hsa-miR-181b | 8.41 | 9.11 | 1.63 |
| SEQ ID NO. 3 | hsa-miR-19a | 7.78 | 8.45 | 1.60 |
| SEQ ID NO. 12 | hsa-miR-95 | 6.12 | 6.79 | 1.59 |
| SEQ ID NO. 62 | hsa-miR-516-3p | 4.87 | 5.52 | 1.57 |
| SEQ ID NO. 25 | hsa-miR-378* | 7.53 | 6.85 | 0.62 |
| SEQ ID NO. 26 | hsa-miR-422b | 10.88 | 10.02 | 0.55 |
| SEQ ID NO. 36 | hsa-miR-422a | 10.05 | 9.19 | 0.55 |
| SEQ ID NO. 24 | hsa-miR-30a-3p | 7.14 | 6.24 | 0.54 |
| SEQ ID NO. 37 | hsa-miR-139 | 7.62 | 6.17 | 0.37 |
| SEQ ID NO. 35 | hsa-miR-195 | 11.04 | 9.52 | 0.35 |
| SEQ ID NO. 28 | hsa-miR-145 | 15.11 | 13.46 | 0.32 |
| SEQ ID NO. 33 | hsa-miR-133a | 10.51 | 7.64 | 0.14 |

### Determining the Stage of a Colorectal Tumor

Additional miRNA sequences have been discovered that are characteristic of late stage (III and IV) colorectal tumors relative to early stage (I and II) tumors. Six miRNA sequences were so identified as differentially regulated in late and early stage colorectal tumors. These sequences are listed in Table 3.

TABLE 3
miRNAs differentially expressed in early (I and II) vs late stage
(III and IV) disease.

| | | Stage | Stage | Fold | |
|---|---|---|---|---|---|
| SEQ ID. | Sample ID | I/II | III/IV | change | p-value |
| SEQ ID NO. 20 | hsa-miR-31 | 7.11 | 9.97 | 7.22 | 1.53E−03 |
| SEQ ID NO. 38 | hsa-miR-7 | 7.77 | 8.85 | 2.11 | 1.96E−02 |
| SEQ ID NO. 39 | hsa-miR-99b | 8.74 | 8.12 | 0.65 | 3.64E−03 |
| SEQ ID NO. 25 | hsa-miR-378* | 6.85 | 6.23 | 0.65 | 3.02E−02 |
| SEQ ID NO. 33 | hsa-miR-133a | 7.64 | 6.96 | 0.63 | 1.64E−02 |
| SEQ ID NO. 31 | hsa-miR-125a | 10.64 | 9.76 | 0.54 | 2.69E−03 |

Fold change = Stage III/IV:Stage I/II

In one embodiment of the invention, total RNA is extracted from a cellular sample. For example, a tissue sample may be removed from a patient during a surgical procedure. Total RNA is extracted from the tissue in accordance with conventional techniques. Small RNA is isolated from the total RNA and thereafter, the abundances of one or more miRNA sequences are observed, relative to a wild type sample. The abundances may be measured with conventional techniques such as, but not limited to, QPCR. Based on the regulatory changes (i.e. up regulation or down regulation relative to a wild type sample) a determination is made as to the stage of cancer. For example, the stage may be determined to be an early stage (I or II) or a late stage (III or IV) cancer. Referring to Table 2, a determination of late stage CRC may be made if a regulation change in one or more of the following miRNAs are observed: hsa-miR-31, hsa-miR-7, hsa-miR-99b, hsa-miR-378, hsa-miR-133a, hsa-miR-125a, or any combination of the aforementioned sequences.

By way of illustration, and not limitation, the regulation change of hsa-miR-31 may be observed. If a significant up regulation is observed, then the sample being tested may be determined to be a late stage colorectal cancer sample. In certain embodiments, such a determination is made only if the magnitude (up regulation or down regulation) and degree (fold change) of regulation change exceeds a specified threshold. For example, in some embodiments a positive diagnosis is made only if there is at least a seven fold up regulation in hsa-miR-31. In another embodiment, a positive diagnosis is made only if there is at least a two fold up regulation in hsa-miR-7. In another embodiment, select sequences such as hsa-miR-99b, hsa-miR-378, hsa-miR-133a, hsa-miR-125a, and combinations thereof, are observed for down regulation. The anticipated degree of down regulation of such sequences is shown in Table 2. Such sequences may be observed individually or in any combination.

In another embodiment, the stage of CRC is determined if more than one miRNA exhibit specified regulatory changes. For example, a determination of a late stage CRC may be made if both (1) hsa-miR-31 exhibits at least a seven fold up regulation and (2) hsa-miR-7 exhibits at least a two fold up regulation. In another embodiment, a late stage CRC is determined to be present if there is an up regulation in hsa-miR-31, hsa-miR-7, or both and such up regulation is accompanied by a down regulation in hsa-miR-99b, hsa-miR-378, hsa-miR-133a, hsa-miR-125a, or in combinations thereof. Threshold criteria, such as a seven-fold up regulation, may be established for each of these miRNA sequences. For example, a threshold criteria of a 0.6 down regulation for has-amiR-133a may be established. The above examples are illustrative only. Any combination of miRNA sequences may be monitored for regulatory changes.

The miRNA may be extracted from a tissue sample, as previously described. Alternatively, miRNA may be isolated from a non-tissue sample. For example, miRNA may be isolated from a blood, stool, urine or other biological sample. The abundance of the specific miRNA found in the sample is compared to a normal sample. Up regulated or down regulated miRNA abundances may be indicative of a cancer.

The miRNA sequences in the attached sequence listing represent commonly isolated miRNA sequences. Alterations at the termini of the listed sequences are known in the art and fall within the scope of the invention provided that the residues are at least 95% homologous.

While the invention has been described with reference to specific embodiments, it will be understood by those skilled in the art that various changes may be made and equivalents may be substituted for elements thereof to adapt to particular situations without departing from the scope of the invention. Therefore, it is intended that the invention not be limited to the particular embodiments disclosed or the particular mode contemplated for carrying out this invention, but that the invention will include all embodiments falling within the scope and spirit of the appended claims.

### Methods

### Cell Lines

SW620, SW480, HCT116 and HT29 cell lines were obtained from the ATCC. KM20L2 and KM12C were provided by the NCI-Frederick Cancer DCT Tumor Repository, while cell lines KM20 and KM12SM were supplied by Dr Isaiah J. Fidler (The University of Texas MD Anderson Cancer Center). SW620 and SW480 cells were grown in Dulbecco's Modified Eagle Medium (D-MEM) (Gibco). HCT116 cells were grown in McCoys 5A Media (Gibco) and KM20, KM20L2, KM12C, KM12SM and HT29 cells were grown in RPMI Media 1640 (Gibco). All media was supplemented with 10% fetal bovine serum (JRH Biosciences), 2 mM L-glutamine (Gibco) and Penicillin-Streptomycin solution (0.1 U/mL penicillin and 0.1 μg/mL streptomycin) (Gibco), except for the HT29 cells which were cultured in 0.72 mM L-glutamine.

### Clinical Samples

In total, 49 snap-frozen human tissue samples were obtained from Genomics Collaborative Inc. (GCI: Cambridge Mass.) or Clinomics Bioscience, Inc (Pittsfield, Mass.), including 4 normal colon, 4 Stage I, 19 Stage II, 20 Stage III and 2 Stage IV (Supplemental Table 1). In addition, 8 matched formalin fixed paraffin embedded (FFPE) samples (3 Stage II, 4 Stage III and 1 Stage IV) were obtained. The median tumor content of all CRC samples was 70%, with no significant difference in tumor content between early stage (I and II) versus late stage (III and IV) disease.

miRNA Profiling

The mirVana Bioarray (Ambion, version 1) that contains 287 human miRNA probes was employed to identify colorectal cancer miRNA signatures. MiRNA was isolated from 5 ug of total RNA from colorectal samples using the mirVana isolation kit (Ambion) for snap-frozen samples and the RecoverAll™ Total Nucleic Acid Isolation. Kit for FFPE samples (Ambion). All samples were then fractionated by polyacrylamide gel electrophoresis (Flash-Page Ambion) and small RNAs (<40 nt) were recovered by ethanol precipitation with linear acrylamide. Quantitative RT-PCR (QPCR) of miR-16 was used to confirm miRNA enrichment prior to miRNA array analysis.

The small RNAs from all samples were subject to poly(A) polymerase reaction wherein amine modified uridines were incorporated (Ambion). The tailed samples were then fluorescently labeled using the amine-reactive Cy3 or Cy5 (Invitrogen). One- or two-color hybridizations were performed for the clinical CRC or cell line profiling experiments, respectively. For 2-color experiments, cell line miRNA was directly compared to normal colon RNA (Ambion). The fluorescently labeled RNAs were purified using a glass-fiber filter and eluted (Ambion). Each sample was then hybridized to the Bioarray slides for 14 hours at 42° C. (Ambion). The arrays were then washed and scanned using an Agilent 2505B confocol laser microarray scanner (Agilent) and data was obtained using the Expression Analysis software (Codelink, version 4.2).

Northern blot analysis of specific miRNAs was performed as follows. TRIzol extraction of total RNA was carried out according to the manufacturer's specifications (Invitrogen). Briefly, cells were washed with PBS and 5 mL TRIzol reagent added and cells incubated for 5 minutes at room temperature. After adding 1 mL chloroform, cells were shaken vigorously for 15 seconds by hand. The samples were centrifuged and the aqueous layer transferred to a 15 mL falcon tube containing 2.5 mL isopropanol. The samples were incubated at room temperature for 20 minutes, centrifuged as above to pellet the RNA and resuspended with 1 mL 75% EtOH. RNA was pelleted by centrifugation, air-dried and resuspended in 50 μL DEPC water (Ambion).

Northern blot analysis was conducted using 15% PAGE-Urea gels, prepared using the SequaGel Sequencing System (National Diagnostics), and electrophoresis was carried out using the MiniProtean II gel electrophoresis apparatus (BioRad). A total of 40 μg RNA was added to 10 μl RNA loading dye (2× solution of 95% Formamide, 18 mM EDTA, and 0.025% SDS, Xylene Cyanol, and Bromophenol Blue) and incubated at 65° C. for 10 minutes. The samples were loaded onto the 15% PAGE-Urea/TBE gel and electrophoresed in 1×TBE at 100V until the bromophenol blue dye reached the bottom of the gel. The RNA was transferred onto the Hybond-N+ membrane (GE Healthcare) using the Mini Trans-Blot Electrophoretic Transfer Cell (BioRad) in 0.5×TBE buffer with 80 V for 1 hour. The RNA was cross-linked to the membrane using the UV Stratalinker 1800 (1200 joules) (Stratagene).

Membranes were pre-hybridized in 10 mL Express hybridization solution (Clontech) at 37° C. The Starfire oligonucleotide probe was boiled for 1 minute and then added to the hybridization solution. After overnight hybridization at 37° C., the hybridization solution was removed and the membrane rinsed three times with 2×SSC/0.1% SDS and further washed with 2×SSC/0.1% SDS solution at 37° C. for 15 minutes. The membrane was exposed to a Storage Phosphor Screen (GE Healthcare) overnight and imaged using the Typhoon Trio machine (GE Healthcare). The membrane was stripped of the bound probe by pouring boiling 0.1% SDS directly onto the membrane and then allowing the solution to slowly cool over a 30 minute period.

Custom Starfire oligonucleotide probes were synthesized by Integrated DNA Technologies (IDT). The lyophilized oligonucleotide probes were diluted to 100 μM stock solution in 1×TE pH 8.0. The labelling reaction included 1×exo⁻ reaction buffer (NEB), 1 μL Starfire Universal template oligonucleotide (IDT) and 0.5 pmol Starfire oligonucleotide probe. The reaction mix was boiled for 1 minute and then allowed to cool to room temperature for 5 minutes before adding 50 μCi α-³²P-dATP (10 mCi/mL, 6000 Ci/mmol) (Perkin-Elmer) and 5 U exo⁻ Klenow DNA polymerase (NEB) and incubating at room temperature for 90 minutes. The reaction was stopped by the addition of 40 μL 10 mM EDTA. The unincorporated α-³²P-dATP was removed from the reaction mix using MicroSpin G-25 columns (GE Healthcare) according to manufacturer's instructions. Prior to use, the probe was boiled for 1 minute.

### Sequences of Starfire Probes Used:

| | |
|---|---|
| miR-1 | |
| 5′ TAC ATA CTT CTT TAC ATT CCA 3′ | SEQ ID NO. 40 |
| miR-126 | |
| 5′ GCA TTA TTA CTC ACG GTA CGA 3′ | SEQ ID NO. 41 |
| miR-19a | |
| 5′ TCA GTT TTG CAT AGA TTT GCA CA 3′ | SEQ ID NO. 42 |
| miR-221 | |
| 5′ GAA ACC CAG CAG ACA ATG TAG CT 3′ | SEQ ID NO. 43 |
| miR-96 | |
| 5′ GCA AAA ATG TGC TAG TGC CAA A 3′ | SEQ ID NO. 44 |
| miR-182 | |
| 5′ TGT GAG TTC TAC CAT TGC CAA A 3′ | SEQ ID NO. 45 |
| miR-145 | |
| 5′ AAG GGA TTC CTG GGA AAA CTG GAC 3′ | SEQ ID NO. 46 |
| miR-30b | |
| 5′ GCT GAG TGT AGG ATG TTT ACA 3′ | SEQ ID NO. 47 |
| miR-194 | |
| 5′ TCC ACA TGG AGT TGC TGT TAC A 3′ | SEQ ID NO. 48 |
| miR-181a | |
| 5′ ACT CAC CGA CAG CGT TGA ATG TT 3′ | SEQ ID NO. 49 |
| miR-143 | |
| 5′ TGA GCT ACA GTG CTT CAT CTC A 3′ | SEQ ID NO. 50 |
| miR-26a | |
| 5′ AGC CTA TCC TGG ATT ACT TGA A 3′ | SEQ ID NO. 51 |
| miR-27a | |
| 5′ GGC GGA ACT TAG CCA CTG TGA A 3′ | SEQ ID NO. 52 |
| miR-103 | |
| 5′ TCA TAG CCC TGT ACA ATG CTG CT 3′ | SEQ ID NO. 53 |
| miR-7 | |
| 5′ AAC AAA ATC ACT AGT CTT CCA 3′ | SEQ ID NO. 54 |
| let-7a | |
| 5′ AAC TAT ACA ACC TAC TAC CTC A 3′ | SEQ ID NO. 55 |
| miR-20a | |
| 5′ CTA CCT GCA CTA TAA GCA CTT TA 3′ | SEQ ID NO. 56 |
| miR-25 | |
| 5′ TCA GAC CGA GAC AAG TGC AAT G 3′ | SEQ ID NO. 57 |
| miR-125b | |
| 5′ TCA CAA GTT AGG GTC TCA GGG A 3′ | SEQ ID NO. 58 |
| miR-155 | |
| 5′ CCC CTA TCA CGA TTA GCA TTA A 3′ | SEQ ID NO. 59 |
| miR-100 | |
| 5′ CAC AAG TTC GGA TCT ACG GGT T 3′ | SEQ ID NO. 60 |

The U6 snRNA oligonucleotide probe (5′ AAC GCT TCA CGA ATT TGC GT 3′, SEQ ID NO. 61) was end labeled using 20 pmole oligonucleotide probe, 1×T4 polynucleotide buffer (NEB), 50 μCi □-³²P-dATP (10 mCi/mL, 6000 Ci/mmol) (Perkin Elmer) and 10 U T4 polynucleotide kinase (NEB), in a final volume of 20 μL. The probe was incubated for 30 minutes at 37° C. The reaction was stopped by the addition of 40 μL, 10 mM EDTA. The unincorporated □-³²P-dATP was removed from the reaction mix using MicroSpin G-25 columns (GE Healthcare) according to manufacturer's instructions. Prior to use, the probe was boiled for 5 minutes.

### Statistical Analysis

The data were analyzed using the R software package. The data were quantile normalized prior to determining differential gene expression. Replicate samples and probe values were averaged and the Student t-test was performed to find genes that vary significantly across sample groups. Genes were selected if the median normalized signal intensity was greater than 100 (75^{th} percentile of median signal) for at least one group, with a mean change >1.5-fold and a p-value <0.05. A one-way ANOVA was used to evaluate miRNA expression level between normal and different cancer stages. Both probe level and gene level data analysis was performed for all group comparisons.

### MiRNA QPCR

QPCR was performed using the ABI miRNA Taqman reagents to verify miRNA expression profiles (Chen, C., Ridzon, D. A., Broomer, A. J., Zhou, Z., Lee, D. H., Nguyen, J. T., Barbisin, M., Xu, N. L., Mahuvakar, V. R., Andersen, M. R., Lao, K. Q., Livak, K. J., and Guegler, K. J. Real-time quantification of microRNAs by stem-loop RT-PCR. Nucleic Acids Res, 33: e179, 2005). Ten ng of total RNA was converted to cDNA using the High Capacity DNA Archive kit and 3u1 of 5×RT primer according to the manufacturer's instructions (Ambion). The 15 μl reactions were incubated in a thermocycler for 30 min at 16° C., 30 min at 42° C., 5 min at 85° C. and held at 4° C. All reverse transcriptase (RT) reactions included no template controls. QPCR was performed using a standard Taqman® PCR kit protocol on an Applied Biosystems 7900HT Sequence Detection System. The 10 μl PCR reaction included 0.66 RT product, 1 μl Taqman microRNA assay primer and probe mix, 5 μl Taqman 2× Universal PCR master mix (No Amperase UNG) and 3.34 n1 water. The reactions were incubated in a 384 well plate at 95° C. for 10 mins, followed by 40 cycles of 95° C. for 15 seq, and 60° C. for 2 min. All QPCR reactions included a no cDNA control and all reactions were performed in triplicate.

What is claimed is:

1. A process for determining the stage of colorectal cancer in humans comprising the steps of:
observing a regulation change of a microRNA from extracted RNA relative to the same microRNA in a wild type colorectal tissue sample, wherein the microRNA is selected from the group consisting of SEQ ID NO. 38, SEQ ID NO. 39, SEQ ID NO. 25, SEQ ID NO. 33, SEQ ID NO. 31, and combinations thereof;
determining the stage of colorectal cancer based on the observed regulation change.

2. The process as recited in claim 1, further comprising the step extracting RNA from a tissue sample prior to the step of observing a regulation change.

3. The process as recited in claim 1, wherein the step of observing a regulation change observes an up regulation in SEQ ID NO. 38.

4. The process as recited in claim 1, wherein the step of observing a regulation change observes a down regulation in a microRNA selected from the group consisting of SEQ ID NO. 39, SEQ ID NO. 25, SEQ ID NO. 33, SEQ ID NO. 31 and combinations thereof.

5. The process as recited in claim 4, wherein the step of observing a regulation change observes a down regulation in SEQ ID NO. 39.

6. The process as recited in claim 4, wherein the step of observing a regulation change observes a down regulation in SEQ 1D NO. 25.

7. The process as recited in claim 4, wherein the step of observing a regulation change observes a down regulation in SEQ ID NO. 33.

8. The process as recited in claim 4, wherein the step of observing a regulation change observes a down regulation in SEQ ID NO. 31.

9. The process as recited in claim 1, wherein the microRNA includes SEQ ID NO. 20 and the step of observing a regulation change observes an up regulation in both SEQ ID NO. 20 and SEQ ID NO. 38.

10. The process as recited in claim 9, wherein the step of observing a regulation change observes at least a seven fold up regulation in SEQ ID NO. 20 and at least a two fold up regulation in SEQ ID NO. 38.

11. The process as recited in claim 9, wherein there is a seven fold or greater up regulation of SEQ ID NO. 20.

12. The process as recited in claim 1, wherein the microRNA includes SEQ ID NO. 38 and the stage of colorectal cancer is determined to be stage III or later if there is a two fold or greater up regulation of SEQ ID NO. 38.

13. A process for diagnosing the stage of colorectal cancer in humans comprising the steps of:
extracting RNA from a colorectal cell;
observing a regulation change of at least two microRNAs from the extracted RNA relative to the same microRNAs in a normal colorectal tissue sample, wherein the microRNAs are selected from the group consisting of SEQ ID NO. 20, SEQ ID NO. 38, SEQ ID NO. 39, SEQ ID NO. 25, SEQ ID NO. 33, SEQ ID NO. 31, and combinations thereof;
determining the stage of colorectal cancer based on the observed regulation change.

14. The process as recited in claim 13, wherein the at least two of the microRNAs include both SEQ ID NO. 20 and SEQ ID NO. 33.

15. The process as recited in claim 14, wherein the stage of colorectal cancer is determined to be stage III or later if
there is a seven fold or greater up regulation in the concentration of SEQ ID NO. 20 in the colorectal tumor relative to a normal colorectal tissue sample; and
there is a 0.6 fold or greater down regulation of SEQ ID NO. 33 in the colorectal tumor relative to a normal colorectal tissue sample.

16. A process for diagnosing the stage of colorectal cancer in humans comprising the steps of:
observing a regulation change of at least two microRNAs that include both SEQ ID NO. 20 and SEQ ID NO. 38;
determining the stage of colorectal cancer based on the observed regulation change.

17. The process as recited in claim 16, wherein the stage of colorectal cancer is determined to be stage III or later if
there is a seven fold or greater up regulation in the concentration of SEQ ID NO. 20 in the colorectal tumor relative to a normal colorectal tissue sample; and
there is a 0.6 fold or greater down regulation of SEQ ID NO. 33 in the colorectal tumor relative to a normal colorectal tissue sample.
